(19) United States
(12) Patent Application Publication
(10) Pub. No.: US 2014/0336735 A1
(43) Pub. Date: Nov. 13, 2014

(54) HEADER CONNECTION WITH REDUCED COMPLEXITY

(71) Applicant: Cardiac Pacemakers, Inc., St. Paul, MN (US)

(72) Inventors: Michael J. Kane, Roseville, MN (US); James Fouhy, Co Cork (IE); Moira B. Sweeney, St. Paul, MN (US)

(73) Assignee: Cardiac Pacemakers, Inc., St. Paul, MN (US)

(21) Appl. No.: 14/276,105

(22) Filed: May 13, 2014

Related U.S. Application Data
(60) Provisional application No. 61/822,646, filed on May 13, 2013.

(51) Int. Cl. A61N 1/375

(52) U.S. Cl. CPC A61N 1/3752
USPC 607/116; 29/825

(57) ABSTRACT

An apparatus includes an implantable housing, a header mounted to the implantable housing and including a connector block cavity, and a connector block located within the connector block cavity, the connector block including a housing portion, a coil spring, and a metallic conductor connected around the coil spring and extending directly to a feedthrough.

### CLAIM OF PRIORITY

This application claims the benefit of priority under 35 U.S.C. §119(e) of U.S. Provisional Patent Application Ser. No. 61/822,646, filed on May 13, 2013, which is incorporated herein by reference in its entirety.

### CROSS-REFERENCE TO RELATED APPLICATION

This application is related to U.S. Provisional Patent Application Ser. No. 61/773,302 to Sweeney, entitled “CONNECTOR BLOCKS FOR A HEADER OF AN IMPLANTABLE DEVICE,” filed Mar. 6, 2013, which is incorporated herein by reference in its entirety.

### BACKGROUND

Implantable medical devices can include cardiac rhythm management devices which commonly include a pacemaker unit which, in turn, includes sensing and control circuitry, together with a power supply, protectively housed in a hermetically sealed case. Such devices can be coupled with one or more electrical leads designed to connect to the patient's heart muscle tissue to facilitate sensing or stimulation of that tissue. To maintain the integrity of the components in the sealed case, a hermetically sealed passage of electrical conductors can be provided to the exterior for connection to the leads and ultimately to tissue. This has been accomplished by using connector blocks and associated feed-through conductors located external to the housing which, themselves, can be placed within a sealed header structure of medical grade polymer material.

While there have been advances in improving the pacemaker devices themselves, the assembly and testing of the devices has remained relatively dependent on the skill of the assembly workers. Attempts have been made in the art to simplify the assembly process and to improve the acceptance rate of completed devices which undergo rigorous testing once assembled. In U.S. Pat. No. 5,282,841 to Szyszkowski, for example, a ribbon conductor set or harness is provided to facilitate connection of feed-through terminal conductors to corresponding connector blocks in the header in which the individual ribbon conductor leads can be shaped to a three-dimensional geometry for ease of assembly. The assembled or fully interconnected unit is thereafter subjected to an overmolding or coating step in which an entire epoxy head is cast over the assembly to encapsulate and seal the connecting components. That system, however, still depends on the skill of workers for assembly and requires extensive overmolding. Furthermore, the electrical integrity of the unit cannot be tested until it is completely assembled.

U.S. Pat. No. 6,205,358 B1 to Haeg et al. discloses a pre-formed header module in combination with a ribbon connector harness which is assembled and placed between feed-through pins on the side of the sealed enclosure and the header module and thereafter captured in an overmolding or backfilling step using medical grade polymer. This system also suffers from similar drawbacks to those described above.

### OVERVIEW

The present inventors have recognized, among other things, that a problem to be solved can include providing a lead connector assembly with a reduced number of joints that can be manufactured easily. The present subject matter can help provide a solution to this problem, such as by providing a metallic conductor that can extend around a spring sized to receive a terminal pin of a lead, the metallic conductor shaped to extend out of a header, along a side of the header, and directly couple to a feedthrough.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an implantable medical device according to an example.

FIG. 2 shows a perspective view of a header core for a header of an implantable medical device, according to an example.

FIG. 3 shows a perspective view of a connector block according to an example.

FIG. 4 shows a perspective view of a connector block according to an example.

FIG. 5 shows a perspective view of a termination member, according to an example.

FIG. 6 shows a perspective view of a termination member, according to an example.

FIG. 7 shows a side cross-section view of the connector block in which a metallic conductor exits a bottom of a connector block, according to an example.

FIG. 8 shows a side cross-section view of the connector block in which a metallic conductor exits a bottom of a connector block and the connector block extends over a cavity, according to an example.

FIG. 9 shows a side cross-section view of the connector block with a side opening for a metallic conductor, according to an example.

FIG. 10 shows a side cross-section view of the connector block with a side opening for a metallic conductor, showing an undeformed metallic conductor superimposed over the connector block, according to an example.

FIG. 11A is a side view of a metallic conductor include leaf springs, according to an example.

FIG. 11B is a front view of a metallic conductor include leaf springs, according to an example.

FIG. 11C is a side cross-section view of the connector block with a metallic conductor include leaf springs, according to an example.

FIG. 12 is a side cross-section view of a metallic conductor coupled to itself, according to an example.

FIG. 13 is a side view of a header core with metallic conductors routed from top (illustrated on the left) to bottom (illustrated on the right), according to an example.

FIG. 14 is a side cross-section view of a metallic conductor of two different thicknesses, according to an example.

FIG. 15 is a method of making a device including a metallic conductor, according to an example.

### DETAILED DESCRIPTION

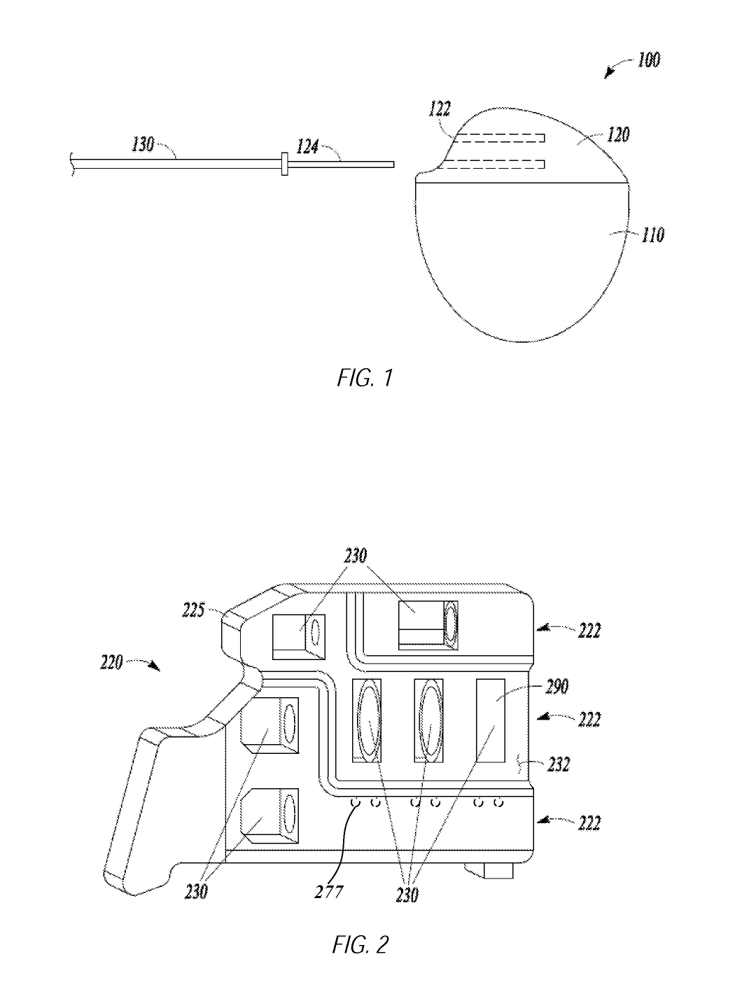

FIG. 1 shows an implantable medical device 100 according to an example. The implantable medical device 100 can include a housing 110, such as a metallic housing. A header 120 can be attached to the housing 110. The header 120 can include one or more ports 122 to receive a terminal pin 124 of an implantable lead 130. The lead 130 can be configured to deliver electronic pulses, such as pacing pulses, defibrillation shock energy, cardioversion therapy to a heart, and the like. The implantable medical device 100 can be implanted in a surgically-formed pocket in a patient's chest or other desired location. The implantable medical device 100 can include electronic components to perform signal analysis, processing, and control. The implantable medical device 100 can include a power supply such as a battery, a capacitor, and other components housed within housing 110. The implantable medical device 100 can include a microprocessor to provide processing and evaluation to determine and deliver electrical shocks and pulses of different energy levels and timing for ventricular defibrillation, cardioversion, and pacing to a heart in response to cardiac arrhythmia including fibrillation, tachycardia, and bradycardia via one or more electrodes of the lead 130. The implantable device 100 can be used to provide neural therapy.

FIG. 2 shows a perspective view of a header core 220 for a header of an implantable medical device, according to an example. The header core 220 can include a core 225, which can be formed of molded plastic. The header core 220 can include one or more terminal pin ports 222 formed in the header core 225 to receive a terminal pin of a lead. The one or more ports 222 can extend longitudinally into the header core 220. The header core 220 can include one or more connector block cavities 230. The cavities 230 can intersect with the terminal pin ports 222. The connector block cavities 230 can open on a side surface 232 of the header core 220. The connector block cavities 230 can be shaped and dimensioned to each receive a connector block 290. The connector block 290 can be side-mounted into the side surface located connector block cavities 230 of the header core 220. The connector block 290 can be connected to one or more feedthroughs that extend from the header into a housing of an implantable device. The feedthroughs can be disposed below the header core oriented as illustrated. Thus, the connector block 290 can receive a lead terminal pin and electrically couple the lead terminal pin to electronics within the housing via the feedthrough.

The header core can include routing guides 277 (channel or boss pin pairs) right into the block

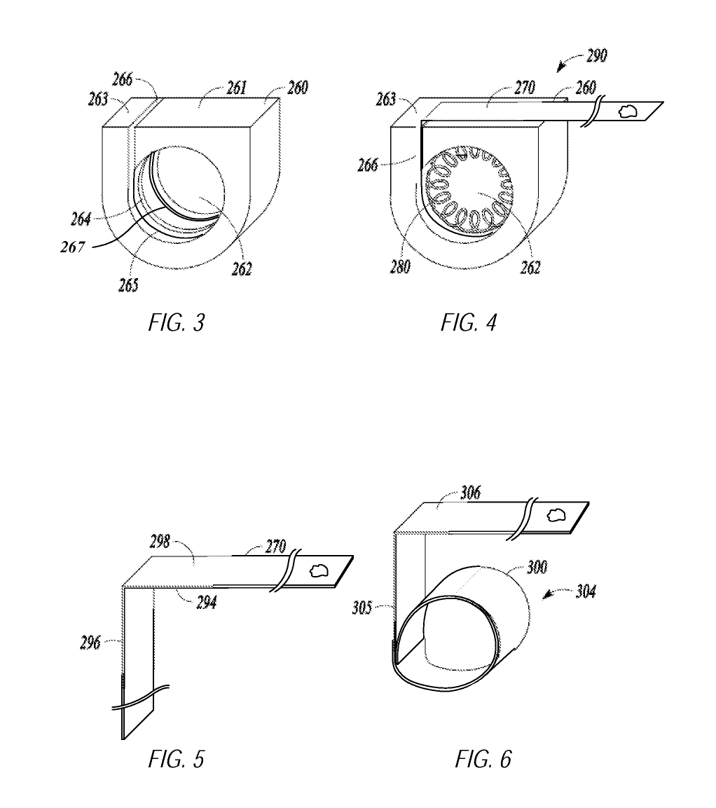

FIG. 3 shows a perspective view of a connector block housing portion 260 according to an example. FIG. 4 shows a perspective view of the connector block 290 according to an example. The housing portion 260 can include a discrete molded body 261 that can be dimensioned to at least partially fit within the connector block cavity 230 (e.g., a cavity of header core illustrated in FIG. 2). The housing portion 260 can be formed of a polymer, and can be pre-molded. The housing portion 260 can include a port 262 configured to receive a spring, such as a coil spring 280, therein, with the spring sized to receive the lead terminal pin. The coil spring can comprise a toroidal shape, for example. Other shapes of the coil spring can also be used. The housing portion 260 can include a spring receiving section 264, such as a race or groove 265, formed in the surface of the housing portion 260 around the periphery of the port 262. In other options, walls or other structures can be formed in the port 262 to receive and hold the spring 280. A channel 267 can be disposed in the connector block portion 260 to receive a metallic conductor, such as to conform to the metallic conductor. The channel can be disposed in a header in instances in which the header receives the spring and metallic conductor directly, e.g., when the connector block assembly forms a monolithic part of a header core.

The housing portion 260 can include a slot 266 that can extend from the port 262 to an exterior of the housing portion 260. The slot 266 can accommodate passage of a metallic conductor 270 that can extend from the spring 262 to an exterior of the housing portion 260. The slot can be sized to form an interference fit with the metallic conductor 270, such as to relieve stress placed on the metallic conductor from other components coupled thereto. The metallic conductor 270 can be formed of stainless steel such MP35N or other suitable metallic materials. The metallic conductor 270 can be connected to the spring 280, extend through the slot 266 and be formed to lie flush on an exposed outer surface 263 of the housing portion 260. Housing portion 260 can include a flat, planar, outer surface 263. The flat, planar outer surface 263 provides for ease of further connections to the metallic conductor 270.

The metallic conductor 270 can be of a length to couple directly to a feedthrough of an implantable housing. Thus, connection can be made between a terminal pin and a feedthrough without intervening couplings, such as solder joints or welds.

FIG. 5 shows a perspective view of the metallic conductor 270, according to an example. The metallic conductor 270 can include a flat ribbon connector 294 formed by stamping, for example. The metallic conductor can include an L-shape with a first end 296 to attach to the coil spring and an upper surface 298 for attachment of the connector 294 to a feedthrough.

FIG. 6 shows a perspective view of a metallic conductor 300, according to an example. The metallic conductor 300 can include a flat ribbon connector 302 formed by stamping, for example. The metallic conductor 300 can include a circular curled portion 304 dimensioned to enclose, such as by encircling, or partially encircling, the spring 280. The metallic conductor 300 can include a straight lead-out section 305 and a flat upper connection surface 306. The metallic conductor 300 can be mounted within the connector block housing portion 260 (FIG. 3) such that the curled portion 304 is within the port 262, the lead-out section 305 extends through the slot 266 and the upper connections surface 306 lies flush against the outer surface 263 of the housing portion 260. The coiled spring 280 is then mounted within the curled portion 304. The metallic conductor 300 can be made to curl upon insertion to a mating connector block. The metallic conductor 300 can be elastically deformable to encircle the spring 280, sandwiched between the header and the spring 280.

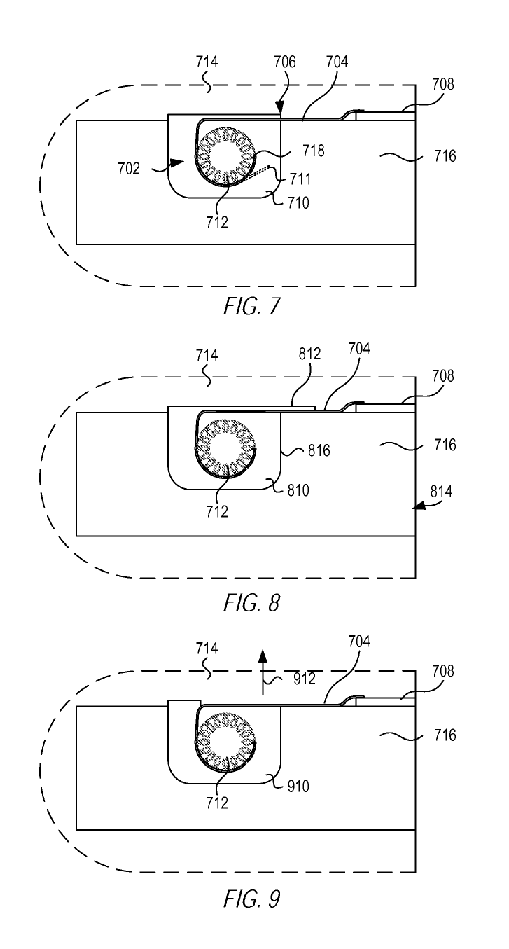

FIG. 7 shows a side cross-section view of the connector block 702 in which a metallic conductor 704 exits a bottom 706 of a connector block 702, according to an example. The metallic connector 704 can connect to a feedthrough 708, according to an example. The connector block 702 can include a connector block housing 710 and a coil spring 712. The metallic conductor 704 can include a flat ribbon connector and can be connected to the spring 712 and can extend to connect directly to the feedthrough 708 which communicates internally to housing 110 illustrated in FIG. 1. This structure can eliminate joining, such as welding, a connector between the metallic conductor 704 and the feedthrough 708. The single weld thus can reduce complexity and increase reliability compared to a one or two joint connection. An overmold 714 can encapsulate the header core in this example. In some embodiments, a pre-molded header can be utilized and the connector blocks can be covered by a medical adhesive sealant.

The illustrated connector block 710 is side-mounted into a header core 716. The metallic conductor 704 is shown extending along the side of the header 716 from the connector block 702 to the feedthrough 708.

As discussed herein, the metallic conductor 704 can be disposed in a header or connector block opening and be made to deform into an opening, and then a channel, to assume a position to surround a spring. A detent 718 or back-stop can block progress of the metallic conductor 704 into the header. Such a detent can be flush with the tangent of the bore diameter as shown or formed to terminate at a remote detent 711 away from the circumference of the bore, which can assist in conductor retention.

FIG. 8 shows a side cross-section view of the connector block in which a metallic conductor exits a bottom of a connector block and the connector block extends over a cavity, according to an example. A housing portion 810 can include a flange 812 that can extend toward a bottom 814 of the header 716. The flange can extend beyond the cavity 816.

FIG. 9 shows a side cross-section view of the connector block with a side opening for a metallic conductor, according to an example. The housing portion 910 can opens laterally 912 to a side of the header 716. The metallic conductor 714 can be used to attach additional components to the circuitry of the device. For example, a resistor can be coupled between metallic conductors of a device. As illustrated, the metallic conductor 704 can bend out of the connector block to extend through the side of the connector block, the header core and to the feedthrough.

In forming a header with the connector block discussed herein, an example can include placing a plastic connector block housing portion into a connector block cavity of a header, placing a coil spring into the connector block housing portion, and connecting a metallic conductor to the coil spring and positioning the metallic conductor to be exposed outside the housing portion. After all the connections have been made between the connector blocks and the feedthroughs of the housing, the header core 716 can be encapsulated with an epoxy overmold 714. The over-mold can be biocompatible and can be disposed over the header and the metallic conductor such that the metallic conductor is disposed between the header and the over-molding layer.

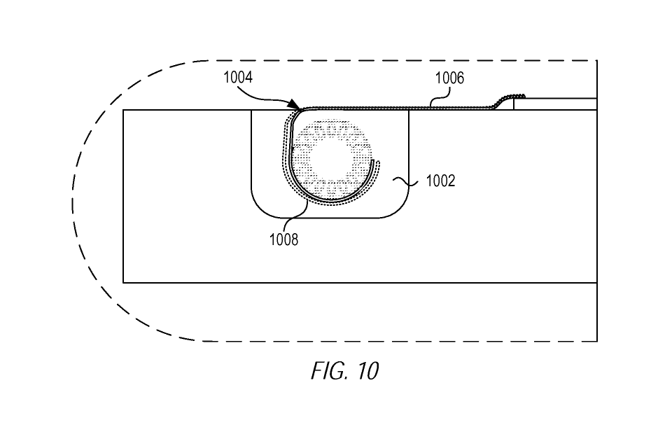

FIG. 10 shows a side cross-section view of the connector block 1002 with a side opening 1004 for a metallic conductor 1006, showing an undeformed metallic conductor 1008 superimposed over the connector block, according to an example. The metallic conductor 1008 can be preformed to a shape larger than the internal channel (e.g., internal channel 267 in FIG. 2), to be elastically deformed to snap-fit into the internal channel.

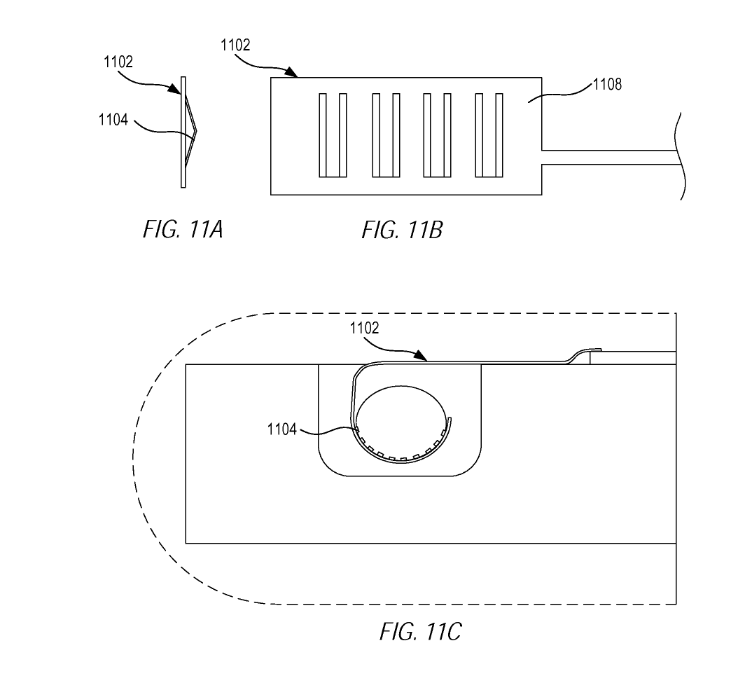

FIG. 11A is a side view of a metallic conductor including leaf springs, according to an example. FIG. 11B is a front view of a metallic conductor including leaf springs, according to an example. FIG. 11C is a side cross-section view of the connector block with a metallic conductor including leaf springs, according to an example. The metallic conductor 1002 can include a plurality of leaf springs 1004 extending toward the terminal pin port 1106. A planar portion 1108 can be stamped, and then rolled into shape. The leaf springs can be formed by stamping.

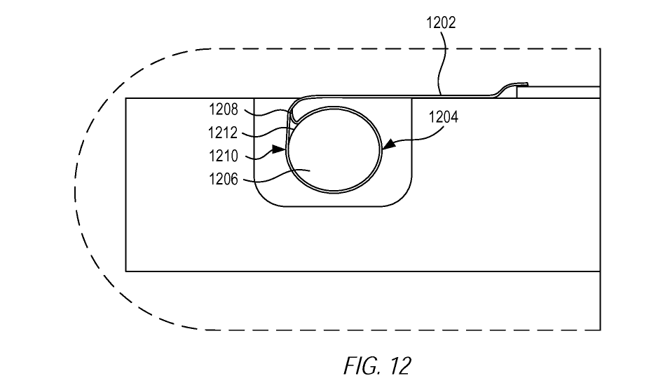

FIG. 12 is a side cross-section view of a metallic conductor coupled to itself, according to an example. A metallic conductor 1202 can form a loop 1204 around the terminal pin port 1206. An end 1208 of the metallic conductor 1202 can be coupled to a center portion 1210 of the metallic conductor 1202. The metallic conductor 1202 can define a gap 1212 between the end 1208 of the metallic conductor 1202 and the center portion 1210 of the metallic conductor 1202, with the spring (not shown) spaced apart from the metallic conductor 1202 along the gap.

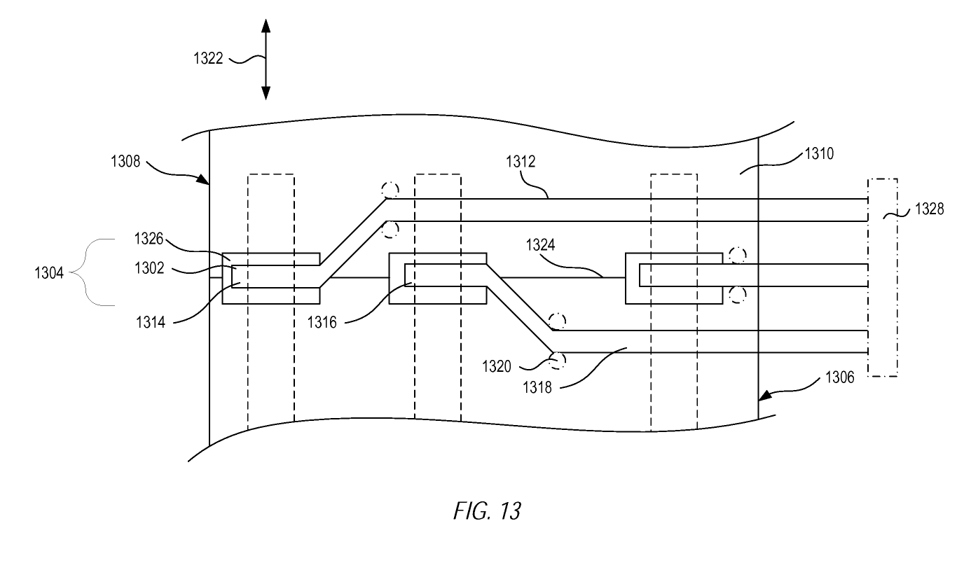

FIG. 13 is a side view of a header core with metallic conductors routed from top (illustrated on the left) to bottom (illustrated on the right), according to an example. A metallic conductor 1312 can be one of a plurality of metallic conductors, each disposed in a header or header core 1310, each routed in separate path extending along a side 1324 of the header core 1310 spaced apart from the others. A plurality of connector blocks 1326 (or springs 1302 if connector blocks are not used) can be aligned in a column 1304 from a bottom 1306 of the header or header core 1310 to a top 1308 of the header 1310. At least one of the metallic conductors, such as an upper metallic conductor 1312, can be pre-formed to route around another metallic conductor. For example, the upper metallic conductor 1312 can be coupled to an upper spring 1314 and can be pre-formed to route around a lower spring 1316 and a lower metallic conductor 1318 coupled to the lower spring 1316. Optional routing guides including, for example, a channel or boss pin pair 1320 (illustrated in dash-dot-dash lines indicating they are optional), can be formed into the header core 1310.

The header block can be assembled axially 1322, out of two-pieces. Accordingly, the metallic conductor can be placed into pre-formed slots in the pieces. A part line 1324 can separate pieces. Such a configuration can reduce the need for the connector blocks.

The plurality of metallic conductors can be formed out of a single stamping. A sacrificial strip 1328 can be used to couple multiple metallic conductors together, and can be removed after the metallic conductors are placed into position, such as in routing guides. The sacrificial strip 1328 can be formed of metal, polymers such as polyimide, tape, such as adhesive tape, and combinations thereof.

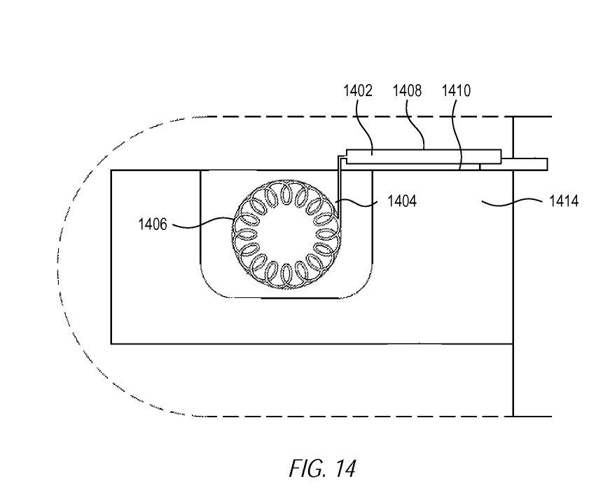

FIG. 14 is a side cross-section view of a metallic conductor of two different thicknesses, according to an example. The metallic conductor 1402 can be comprised of a multi-thickness conductor. A conductor can have multiple thicknesses, with a first thickness 1404 extending around the spring 1406, and a second thickness 1408 extending along an exterior 1410 of the header 1414. The conductor can be wire or ribbon shaped. In some examples, a smaller portion of the wire can be used to form the coil wire. A thicker portion used can be used as a pigtail. Such a configuration can provide for less strain at transition from one portion of the assembly to another. Examples can be tapered from one size to another, forming a wedge or ramp shape.

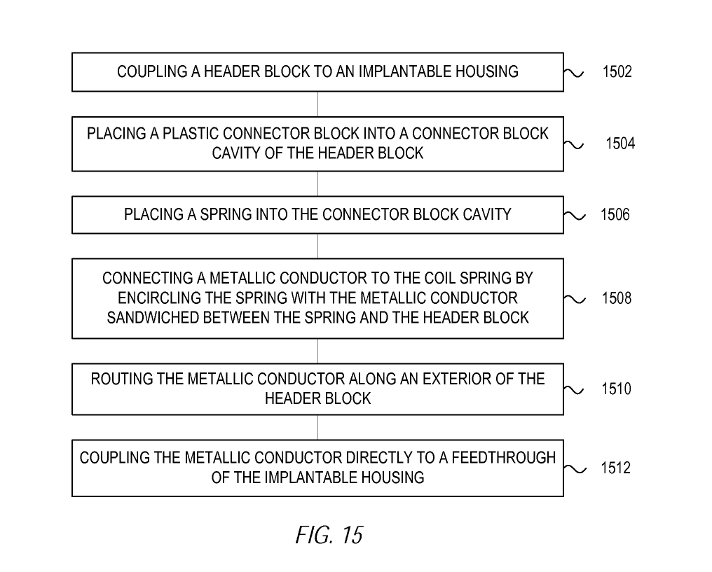

FIG. 15 is a method of making a device including a metallic conductor, according to an example. At 1502, the method can include coupling a header core to an implantable housing. At 1504, the method can include placing a plastic connector block into a connector block cavity of the header core. At 1506, the method can include placing a spring into the connector block cavity. At 1508, the method can include connecting a metallic conductor to the spring by encircling the spring with the metallic conductor sandwiched between the spring and the header core. At 1510, the method can include routing the metallic conductor along an exterior of the header core. At 1512, the method can include coupling the metallic conductor directly to a feedthrough of the implantable housing.

Methods can include overmolding the header with an epoxy. A method can include connecting the metallic conductor includes elastically deforming the metallic conductor while inserting the metallic conductor into an opening on a side of the connector block that is substantially coplanar with a side of the header core. A method can include inserting the metallic conductor includes inserting until the metallic conductor abuts a backstop of the connector block.

### Various Notes & Examples

Example 1 can include or use subject matter (such as an apparatus, a method, a means for performing acts, or a device readable medium including instructions that, when performed by the device, can cause the device to perform acts), such as can include or use an implantable housing including a feedthrough. The Example can include a header coupled to the implantable housing, the header being biocompatible and defining a cavity intersecting with a terminal pin port extending longitudinally into the header. The Example can include a spring disposed in the cavity in a circular configuration encircling the terminal pin port and defining an at least partial spring circumference. The Example can include a metallic conductor that is elastically deformable and conformed to the cavity around the spring along the at least partial spring circumference, the deformable metallic conductor sandwiched between the header and the spring and extending through the header to define a first connection terminal coupled to the feedthrough.

Example 2 can include, or can optionally be combined with any portion or combination of any portions of any one or more of the previous examples, comprising a connector block disposed in the cavity, with the metallic conductor and the spring disposed in the connector block, with the metallic conductor extending through the connector block.

Example 3 can include, or can optionally be combined with any portion or combination of any portions of any one or more of the previous examples, wherein the metallic conductor extends through an opening in the connector block, interference fit through the opening.

Example 4 can include, or can optionally be combined with any portion or combination of any portions of any one or more of the previous examples, wherein a side the connector block extends beyond a side of the header, and defines an opening through a bottom of the connector block, with the metallic conductor extending through the opening.

Example 5 can include, or can optionally be combined with any portion or combination of any portions of any one or more of the previous examples, wherein a side the connector block extends beyond a side of the header, and defines an opening through a bottom of the connector block, with the metallic conductor extending through the opening.

Example 6 can include, or can optionally be combined with any portion or combination of any portions of any one or more of the previous examples, wherein the connector block includes a flange that extends toward a bottom of the header, beyond the cavity.

Example 7 can include, or can optionally be combined with any portion or combination of any portions of any one or more of the previous examples, wherein the connector block cavity opens laterally to a side of the header, wherein the connector block is side-mounted into the header defining an opening in the side of the connector block, with the metallic conductor extending through the opening and bending to extend along the side of the connector block and the header to the feedthrough.

Example 8 can include, or can optionally be combined with any portion or combination of any portions of any one or more of the previous examples, wherein the connector block cavity opens laterally to a side of the header, wherein the connector block is side-mounted into the header, with the metallic conductor extending along the side of the header from the connector block to the feedthrough.

Example 9 can include, or can optionally be combined with any portion or combination of any portions of any one or more of the previous examples, wherein the connector block includes an internal channel shaped to receive the metallic conductor.

Example 10 can include, or can optionally be combined with any portion or combination of any portions of any one or more of the previous examples, wherein the metallic conductor is preformed to a shape larger than the internal channel, to be elastically deformed to snap-fit into the internal channel.

Example 11 can include, or can optionally be combined with any portion or combination of any portions of any one or more of the previous examples, comprising an over-molded layer that is biocompatible and disposed over the header and the metallic conductor such that the metallic conductor is disposed between the header and the over-molding layer.

Example 12 can include, or can optionally be combined with any portion or combination of any portions of any one or more of the previous examples, wherein the metallic conductor comprises a coil spring defining a toroidal shape extending around the terminal pin port.

Example 13 can include, or can optionally be combined with any portion or combination of any portions of any one or more of the previous examples, wherein the metallic conductor encircles the spring.

Example 14 can include, or can optionally be combined with any portion or combination of any portions of any one or more of the previous examples, wherein the metallic conductor comprises a flat ribbon connector.

Example 15 can include, or can optionally be combined with any portion or combination of any portions of any one or more of the previous examples,

wherein the conductor has multiple thicknesses, with a first thickness extending around the spring, and a second thickness extending along an exterior of the header.

Example 16 can include, or can optionally be combined with any portion or combination of any portions of any one or more of the previous examples, wherein the metallic conductor includes a plurality of leaf springs extending toward the terminal pin port.

Example 17 can include, or can optionally be combined with any portion or combination of any portions of any one or more of the previous examples, wherein the metallic conductor forms a loop around the terminal pin port.

Example 18 can include, or can optionally be combined with any portion or combination of any portions of any one or more of the previous examples, wherein an end of the metallic conductor is coupled to a center portion of the metallic conductor.

Example 19 can include, or can optionally be combined with any portion or combination of any portions of any one or more of the previous examples, wherein the metallic conductor defines a gap between the end of the metallic conductor and the center portion of the metallic conductor, with the spring spaced apart from the metallic conductor along the gap.

Example 20 can include, or can optionally be combined with any portion or combination of any portions of any one or more of the previous examples, wherein the metallic conductor is one of a plurality of metallic conductors disposed in the header, each routed in separate path extending along a side of the header spaced apart from the others.

Example 21 can include, or can optionally be combined with any portion or combination of any portions of any one or more of the previous examples, wherein a plurality of springs are aligned in a column from a bottom of the header to a top of the header, and at least one of the metallic conductors coupled to an upper spring is pre-formed to route around a lower spring and a lower metallic conductor coupled to the lower spring.

Example 22 can include or use subject matter (such as an apparatus, a method, a means for performing acts, or a device readable medium including instructions that, when performed by the device, can cause the device to perform acts), such as can include or use and implantable housing including a feedthrough. The Example can include a header core coupled to the implantable housing, the header core being biocompatible and defining a cavity intersecting with a terminal pin port extending longitudinally into the header core. The Example can include a connector block disposed in the cavity with the terminal pin port extending into the connector block. The Example can include a spring disposed in the connector block in a circular configuration encircling the terminal pin port and defining an at least partial spring circumference. The Example can include a metallic conductor that is elastically deformable and conformed to the cavity around the spring along the at least partial spring circumference, the deformable metallic conductor sandwiched between the connector block and the spring and extending through the connector block to define a first connection terminal coupled to the feedthrough, wherein the header core is overmolded with an epoxy to encapsulate the connector block and the header core.

Example 23 can include, or can optionally be combined with any portion or combination of any portions of any one or more of the previous examples, comprising an overmold encapsulating the header core, the connector block, the spring and the metallic conductor.

Example 24 can include, or can optionally be combined with any portion or combination of any portions of any one or more of the previous examples, wherein the header core is formed of a polymer.

Example 25 can include, or can optionally be combined with any portion or combination of any portions of any one or more of the previous examples, wherein the header is pre-molded.

Example 26 can include, or can optionally be combined with any portion or combination of any portions of any one or more of the previous examples, wherein the connector block is formed of a polymer.

Example 27 can include, or can optionally be combined with any portion or combination of any portions of any one or more of the previous examples, wherein the connector block is pre-molded.

Example 28 can include or use subject matter (such as an apparatus, a method, a means for performing acts, or a device readable medium including instructions that, when performed by the device, can cause the device to perform acts), such as can include or use a method including coupling a header core to an implantable housing. The Example can include placing a plastic connector block into a connector block cavity of the header core. The Example can include placing a spring into the connector block cavity. The Example can include connecting a metallic conductor to the spring by encircling the spring with the metallic conductor sandwiched between the spring and the header core. The Example can include routing the metallic conductor along an exterior of the header core. The Example can include coupling the metallic conductor directly to a feedthrough of the implantable housing.

Example 29 can include, or can optionally be combined with any portion or combination of any portions of any one or more of the previous examples, wherein the metallic conductor is non-circumferential around the spring.

Example 30 can include, or can optionally be combined with any portion or combination of any portions of any one or more of the previous examples,

including overmolding the header with an epoxy.

Example 31 can include, or can optionally be combined with any portion or combination of any portions of any one or more of the previous examples, including connecting the metallic conductor includes elastically deforming the metallic conductor while inserting the metallic conductor into an opening on a side of the connector block that is substantially coplanar with a side of the header core.

Example 32 can include, or can optionally be combined with any portion or combination of any portions of any one or more of the previous examples, wherein inserting the metallic conductor includes inserting until the metallic conductor abuts a backstop of the connector block.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The invention claimed is:

1. An apparatus, comprising:
an implantable housing including a feedthrough;
a header coupled to the implantable housing, the header defining a cavity intersecting with a terminal pin port extending longitudinally into the header;
a spring disposed in the cavity in an at least semi-circular configuration at least partially encircling the terminal pin port and defining an at least partial spring circumference; and
a metallic conductor that is elastically deformable and conformed to the cavity around at least a portion of the spring along the at least partial spring circumference, the deformable metallic conductor sandwiched between the header and the spring and extending through the header to define a first connection terminal coupled to the feedthrough.

2. The apparatus of claim 1, comprising a connector block disposed in the cavity, with the metallic conductor and the spring disposed in the connector block, with the metallic conductor extending through the connector block.

3. The apparatus of claim 2, wherein a side of the connector block extends beyond a side of the header, and defines an opening through a bottom of the connector block, with the metallic conductor extending through the opening.

4. The apparatus of claim 2, wherein a side of the connector block extends beyond a side of the header, and defines an opening through a bottom of the connector block, with the metallic conductor extending through the opening.

5. The apparatus of claim 4, wherein the connector block includes a flange that extends toward a bottom of the header, beyond the cavity.

6. The apparatus of claim 2, wherein the connector block cavity opens laterally to a side of the header, wherein the connector block is side-mounted into the header, with the metallic conductor extending along the side of the header from the connector block to the feedthrough.

7. The apparatus of claim 1, wherein the header includes an internal channel shaped to receive the metallic conductor and the metallic conductor is preformed to a shape larger than the internal channel, to be elastically deformed to snap-fit into the internal channel.

8. The apparatus of claim 1, wherein the metallic conductor comprises a flat ribbon connector.

9. The apparatus of claim 8, wherein the conductor has multiple thicknesses, with a first thickness extending around the spring, and a second thickness extending along an exterior of the header.

10. The apparatus of claim 1, wherein the metallic conductor includes a plurality of leaf springs extending toward the terminal pin port.

11. The apparatus of claim 1, wherein the metallic conductor forms a loop around the terminal pin port.

12. The apparatus of claim 1, wherein the metallic conductor is one of a plurality of metallic conductors disposed in the header, each routed in a separate path extending along a side of the header spaced apart from the others.

13. A method comprising:
coupling a header core to an implantable housing;
placing a plastic connector block into a connector block cavity of the header core;
placing a spring into the connector block cavity;
connecting a metallic conductor to the spring by encircling the spring with the metallic conductor sandwiched between the spring and the header core;
routing the metallic conductor along an exterior of the header core; and
coupling the metallic conductor directly to a feedthrough of the implantable housing.

14. The method of claim 13, wherein connecting the metallic conductor includes elastically deforming the metallic conductor while inserting the metallic conductor into an opening on a side of the connector block that is substantially coplanar with a side of the header core.

15. The method of claim 14, wherein inserting the metallic conductor includes inserting until the metallic conductor abuts a backstop of the connector block.

16. An apparatus, comprising:
an implantable housing including a feedthrough;
a header core coupled to the implantable housing, the header core being biocompatible and defining a cavity intersecting with a terminal pin port extending longitudinally into the header core;
a connector block disposed in the cavity with the terminal pin port extending into the connector block;
a spring disposed in the connector block in a circular configuration encircling the terminal pin port and defining an at least partial spring circumference;
a metallic conductor that is elastically deformable and conformed to the cavity around the spring along the at least partial spring circumference, the deformable metallic conductor sandwiched between the connector block and the spring and extending through the connector block to define a first connection terminal coupled to the feedthrough, wherein the header core is overmolded with an epoxy to encapsulate the connector block and the header core.

17. The apparatus of claim 16, comprising an overmold encapsulating the header core, the connector block, the spring and the metallic conductor.

18. The apparatus of claim 16, wherein the header core is formed of a polymer.

19. The apparatus of claim 18, wherein the header is pre-molded.

20. The apparatus of claim 16, wherein the connector block is formed of a polymer.
